(19) United States
(12) Patent Application Publication
(10) Pub. No.: US 2006/0160156 A1
(43) Pub. Date: Jul. 20, 2006

(54) Tears as a screening medium

(76) Inventors: Suzanne Klimberg, Little Rock, AR (US); Larry Suva, Little Rock, AR (US)

Correspondence Address: Benjamin Aaron Adler;ADLER & ASSOCIATES, 8011 Candle Lane, Houston, TX, 77071, US

(21) Appl. No.: 11/332,702

(22) Filed: Jan. 13, 2006

Related U.S. Application Data
(60) Provisional application No. 60/643,489, filed on Jan. 13, 2005.

(51) Int. Cl. G01N 33/574; G01N 33/00

(52) U.S. Cl. 435/7.23; 436/86

(57) ABSTRACT

The present invention is directed to the use of surface-enhanced laser desorption/ionization time-of-flight mass spectrometry (SELDI-TOF-MS) on tears of breast cancer patients. The present invention demonstrates the use of this highly sensitive and specific method to identify proteins and protein patterns in tears that show promise as a diagnostic indicator. Based on this demonstration, the present invention provides a novel, easy and non-invasive screening tool for detecting breast cancer and assessing treatment response.

### CROSS-REFERENCE TO RELATED APPLICATION

This non-provisional application claims benefit of provisional application U.S. Ser. No. 60/643,489, filed Jan. 13, 2005, now abandoned.

### BACKGROUND OF THE INVENTION

1. Field of the Invention

The present invention relates generally to the field of diagnosis and identification of medical conditions and syndromes. More specifically, the present invention involves using tears as a screening medium for the diagnosis and identification of medical conditions and syndromes, including cancer, e.g., breast cancer.

2. Description of the Related Art

Every woman is at risk for breast cancer. Other than skin cancer breast cancer is the most common cancer among women, and accounts for nearly one third of cancers in women in the U.S. In 2000, there were approximately 183,000 new cases of invasive breast cancer that were diagnosed in addition to 40,000 new incidences of ductal carcinoma in situ (DCIS). In the past year alone, over 43,000 women died from breast malignancies.

The key to fighting and curing breast cancer is early detection. According to the data provided by the American Cancer Society, the five-year survival of women in whom the disease is detected at localized stage is greater than 96%. However, the five-year survival decreases to 77% and 21% in women with regional and distant disease, respectively. Between 1950 and the late 1980s, the overall mortality due to breast cancer was relatively stable. But since 1989 the death rates due to this cancer have decreased 1.8% per year on average (Ries L. A. G. et al., 1999). This decline in breast cancer mortality has been attributed to improvements in treatments and benefits of mammography screening. Specifically, increased use of mammography has led to the diagnosis of smaller and more easily treatable cancers. Between 1982 and 1988, the incident rates more than doubled for tumors smaller than 2 cm and decreased by 27% for tumors more than 3 cm. Additionally, the ductal carcinoma in situ rates increased dramatically by approximately 28% in 1982-1988 and by a further 6% per year through 1996 (Garfinkel L. et al., 1994). Thus, the shift in stage of disease at diagnosis towards earlier, more curable cancers improved survival and reduced mortality.

Despite this encouraging progress in earlier detection, only 62% of breast cancers diagnosed between 1989 and 1995 were found while still localized (Ries L. A. G. et al., 1999). Mammography is an accurate detection method, identifying approximately 90% of cancers in asymptomatic women. However, not all women who should be receiving mammograms are doing so on a yearly basis. The median percentage of women in the US in 1997 who had a mammogram within the previous year was 58.4% in the age group of 50 years and older and 55.1% in the age group of 65 years and up. However, these rates varied by state. Arkansas had lowest compliance at 43.3% and 38.9% for these age groups, respectively (Ardekani et al., 2002). Thus, not only are women not undergoing annual breast cancer screening, but also those that are least likely to go for mammography are the ones at the greatest risk for malignancy since the incidence of breast cancer increases with age. The reasons cited by the women for not getting mammograms include discomfort, lack of insurance and difficulties with access.

Serum-based screening tools enhance existing methods of cancer detection and have shown promise in detection of ovarian (Ardekani A. M. et al., 2002), prostate (Miller T. E. et al., 1992), colon and breast (Luftner D. and Possinger K., 2002) cancers. Most of these tests used a combination of liquid chromatography and surface-enhanced laser desorption/ionization time-of-flight mass spectrometry (SELDI-TOF-MS) to analyze serum. However due to presence of large numbers of proteins in the samples from blood, these techniques have become tedious and difficult not only to perform but also to interpret. In addition, important proteins may be lost in the necessary sample preparation needed to run blood on surface-enhanced laser desorption/ionization time-of-flight mass spectrometry. Therefore, there is a need for a screening medium to enhance existing methods of detecting cancer.

Tears are the filtered product of blood and epithelial markers. Hence, markers such as those found in the breast are also found in tears (Schulz B. L. et al., 2002). Unlike serum, surface-enhanced laser desorption/ionization mass spectrometry of tears can be performed directly on tears and requires no processing or separation. Additionally, collection of tears is also easy and non-invasive. Despite this, with the exception of a single case report on HIV patients, which showed distinctive results (Burtin T. et al., 1998), there are no reports on the use of surface-enhanced laser desorption/ionization time-of-flight mass spectrometry on tears of cancer patients.

Thus, prior art is deficient in an approach to screen for cancer, which is non-invasive and easy to perform and interpret. Further, novel proteins and/or novel protein patterns found by surface-enhanced laser desorption/ionization time-of-flight mass spectrometry would easily be translated into a test for persons interested in screening for breast cancer and other cancers. The present invention fulfills this long-standing need and desire in the art.

### SUMMARY OF THE INVENTION

The present invention provides a novel, non-invasive and easy screening tool for diseases such as cancer, including but not limited to breast cancer, by identifying proteins and protein patterns in the tears of patient with breast cancer using surface-enhanced laser desorption/ionization time-of-flight mass spectrometry.

In the present invention, lachrymal fluid from individuals with breast cancer and those without breast cancer were collected. The protein pattern profiles in these unprocessed samples were compared using surface-enhanced laser desorption/ionization time-of-flight mass spectrometry. This procedure identified proteins capable of being used as a diagnostic indicator of breast cancer based on the comparison of their peak intensities in these samples.

In one embodiment of the present invention provides a method for identifying proteins useful as a diagnostic indicator of cancer in an individual. This method comprises obtaining a biological sample from the individual. This sample is then subjected to surface-enhanced laser desorption/ionization time-of-flight mass spectrometry. The protein profile thus obtained is then compared with the protein profile obtained likewise from a control sample. This is followed by selection of proteins in the biological sample that have a statistically greater peak intensities than the peak intensities of the proteins in the control sample, thereby identifying proteins that can serve as diagnostic indicators of the cancer being investigated. In a related embodiment such SELDI-MS profiles can be used as a fingerprint for diagnosis of cancer.

In another embodiment the present invention encompasses proteins identified by the method described above.

In still another embodiment of the present invention is a method for screening an individual for cancer. This method comprises obtaining a biological sample from the individual. The amount(s) of protein(s) claimed above is determined in the biological sample. This amount(s) is then compared to the amount(s) of the protein(s) in control sample, where increased amount(s) of the protein(s) in the biological sample compared to the control sample indicates that the individual has cancer.

In yet another embodiment, the present invention provides a kit for diagnosis of cancer comprising one or more proteins identified by the screening method described above, antibodies to these proteins and other reagents required to perform an immunological assay.

Other and further aspects, features, and advantages of the present invention will be apparent from the following description of the presently preferred embodiments of the invention. These embodiments are given for the purpose of disclosure

### BRIEF DESCRIPTION OF THE DRAWINGS

So that the matter in which the above-recited features, advantages and objects of the invention, as well as others, which will become clear, are attained and can be understood in detail, more particular descriptions of the invention are briefly summarized. The above may be better understood by reference to certain embodiments thereof, which are illustrated in the appended drawings. These drawings form a part of the specification. It is to be noted; however, that the appended drawings illustrate preferred embodiments of the invention and therefore are not to be considered limiting in their scope.

FIGS. 1A-B show results of surface-enhanced laser desorption/ionization time-of-flight mass spectrometry performed using lachrymal fluid of normal individuals and those with breast cancer (protein identification range 1-10 kDa). FIG. 1A shows the results in the form of peak intensities. FIG. 1B shows the same results in the form of bands (akin to a gel) to visualize the relative concentration of each protein.

FIGS. 2A-B show results of surface-enhanced laser desorption/ionization time-of-flight mass spectrometry performed using lachrymal fluid of normal individuals and those with breast cancer (protein identification range 1-10 kDa). FIG. 1A shows the results in the form of peak intensities. FIG. 1B shows the same results in the form of bands (akin to a gel) to visualize the relative concentration of each protein.

FIG. 3A shows the results of surface-enhanced laser desorption/ionization time-of-flight mass spectrometry performed using lachrymal fluid of normal individuals and those with breast cancer which identifies a 35 kDa protein that is over expressed in individuals with invasive and non-invasive breast cancer. FIG. 3B is a rank graph showing the peak intensities for the proteins identified in the SELDI spectrum. The diamonds in red represent peak intensities for the 35 kDa protein in normal patients and the blue squares represent the same in cancer patients.

### DETAILED DESCRIPTION OF THE INVENTION

Although surface-enhanced laser desorption/ionization time-of-flight mass spectrometry had been used to profile serum protein patterns, its use in profiling protein patterns in tears as described in the present invention is novel. The present invention also demonstrates that human tear fluid contains proteins that either individually as biomarkers or collectively as a biosignature can be used as a diagnostic indicator of breast cancer or other cancers. Additionally, the use of tears in the analysis of proteins significant as biomarkers in breast cancer detection has several advantages. First, the collection of tears for the analysis is relatively easy and non-invasive. Secondly, surface-enhanced laser desorption/ionization time-of-flight mass spectrometry can be performed directly on the tears and requires no sample separation or processing as is required in the case of other biological samples such as blood serum or other tissue fluids.

The present invention compares the protein profile in tear fluid from breast cancer patients with those without breast cancer for differences that identify certain proteins as diagnostic indicators of the disease. The present invention also contemplates evaluating the usefulness of proteins identified or protein patterns (signature) found in tears besides their use in diagnosis of cancer. In addition to developing a screening tool for the detection of breast cancer, the present invention also contemplates using tears to assess treatment response to breast cancer. Further, it is also contemplated that this screening technique could be used in the future with the same ease as currently used pregnancy tests. Similar to the pregnancy tests, placing a small amount of lachrymal fluid on test paper and comparing with appropriate controls would enable a woman to obtain a diagnostic result at home with great ease

The present invention is directed to a method of identifying proteins useful as a diagnostic indicator of cancer in an individual, comprising: obtaining a biological sample from the individual; performing surface-enhanced laser desorption/ionization time-of-flight mass spectrometry on the biological sample; comparing a protein profile in the sample with a protein profile in control sample; and selecting proteins in the biological sample having statistically greater peak intensities than the peak intensities of the proteins in the control sample, thereby identifying the proteins as diagnostic indicators of the cancer being investigated. Generally, the proteins identified by this method have molecular weights of about 1-100 kDa. Specifically, the biological sample and the control sample is tear fluid. More specifically, the control sample is obtained from a healthy individual whereas the biological sample is obtained from an individual either diagnosed with or suspected of having cancer. Specifically, the diagnostic indicators detected by this method are for breast cancer. More specifically, the protein identified by the method described supra in the case of breast cancer has a molecular weight of about 35 kDa. The method described supra also identified three proteins in the tear sample from individuals with invasive or non invasive breast cancer, which were present at a concentration lower than that present in tear samples from normal individuals.

In a related embodiment the protein profile or protein signature obtained by SELDI-MS can also be used to diagnose cancer in an individual. In this method, the need to specifically identify diagnostic markers or each individual protein with differential expression in the diseased state is eliminated. This method just requires comparison of the SELDI-MS tear profile of test individual with a control SELDI-MS tear profile typical for the type of cancer being investigated to diagnose if the test individual has that cancer. A match between the test and control profiles indicates that the test individual has cancer. For example, the SELDI-MS profiles obtained from the tear samples of individuals with breast cancer can be used to generate a protein based fingerprint profile typically obtained for breast cancer. The SELDI-MS profile obtained from a test individual can then be compared with this protein fingerprint profile for breast cancer. A match of the test profile with the control fingerprint profile indicates that the test individual has breast cancer. Thus this method eliminates the additional step of having to quantify protein markers to diagnose a diseased state. Signature profiles obtained by MS are very specific for a given biological sample. Such profiles because of their high specificity have been used earlier on to identify different species of bacteria (Dickinson D. N. et al., 2004) The present invention is also directed to proteins identified by the method claimed above. All other aspects regarding the size of the proteins, type of biological sample, the individual and the type of cancer is as discussed above.

The present invention is further directed to a method of screening an individual for cancer, comprising: obtaining biological sample from the individual; determining amount(s) of protein(s) claimed above in the biological sample; and comparing the amount(s) to the amount(s) of protein(s) in control sample, where increased amount(s) of the protein(s) in the biological sample compared to the control sample indicates that the individual has cancer.

This method further comprises assessing a response to a treatment for cancer in an individual. This assessment step comprises: comparing the amount(s) of protein(s) in the sample of the individual collected post-treatment with the amount(s) of the protein(s) in a control sample or in the sample of the individual collected pre-treatment, where the amount(s) of the protein(s) collected post-treatment if same as the amount(s) of the protein(s) in the control sample or decreased compared to the amount(s) of the protein(s) collected pre-treatment indicates that the individual is responsive to the treatment.

All other aspects regarding the type of biological and control samples and cancer is the same as described above. In general, samples of individuals either suspected of having cancer, at risk of developing cancer or being treated for cancer can be screened using the method described above. Individuals suspected of having cancer or at risk of developing cancer are identified based on clinical symptoms such as an abnormal mammogram/ultrasound or a palpable lump or a benign breast evaluation, genetic predisposition and/or lifestyle. Additionally, the amount of protein can be determined by using immunoassays that are known in the art. The most commonly used immunoassays are ELISA, radioimmunoassay and agglutination inhibition reaction.

Further, the present invention also contemplates a kit for screening tears for cancer, including but not limited to breast cancer. This kit can be used with the same ease as the pregnancy kit. This kit will comprise protein(s) identified, an antibody to the protein(s) identified and reagents to detect the binding of the antibody to the protein. Preferably, the biological sample that is tested with this kit is a tear sample. The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Study Design

Cases with malignancy and controls matched for race, sex and menopausal status were chosen. The participants of this study were 18 years of age or older and were seen for either a benign breast evaluation or evaluation of an abnormal mammogram/ultrasound or a palpable lump. Tears were collected 1 week (±4 days) prior to biopsy and 6 months after excisional surgery (lumpectomy or mastectomy). For those women with benign biopsies, the schedule remained the same since this was the standard procedure for women with a history of breast conditions requiring biopsy. The criteria used to include or exclude participants in the study is as shown in the Table 1.

TABLE 1
Inclusion and Exclusion Criteria

| Inclusion Criteria | Exclusion Criteria |
|---|---|
| Female, 18-100 years old | Less than 18 years or more than |
| | 100 years old. |
| Participant is pre-biopsy for a breast | Concurrent eye infection or trauma |
| condition | |
| Suspected lesion is primary or | Active conjunctivitis |
| recurrent | |
| | Abnormal production of tears (too |
| | much or too little) |

### EXAMPLE 2

Study Procedure

A tear sample (˜75 microliters) of the participant was collected in a tuberculin syringe without the needle at the initial clinic visit. In case of participants with an abnormal (suspicious) mammogram/ultrasound or a palpable lump who have undergone a biopsy or surgery, another tear sample was collected at the time of their 1^{st} post-operative visit (1 week). In case of participants undergoing breast surgery (benign or malignant), a third and final tear sample was collected at 6 months during a follow-up appointment.

The sample collected was put directly on ice. In cases where tears are not produced naturally, a natural substance (e.g. onions) instead of a formal lachrymator (gas, chemical substance) would be used to induce production. This natural substance would not have an effect on the result of surface-enhanced laser desorption/ionization time-of-flight mass spectrometry analysis and would not be harmful to the patient.

The tear samples thus collected were diluted 1:5 fold in phosphate-buffered saline (PBS) and applied in duplicate onto each well of a 192-well bioprocessor containing 16-spot NP20 chips (Ciphergen). The bioprocessor was then sealed and incubated with the samples for an hour with vigorous agitation on a Micromix 5 platform shaker. A pooled QC sample prepared in the same manner was applied to duplicate spots on each chip used in each experiment as reproducibility control. The excess mixtures were discarded and the chips were washed three times with PBS. The chips were then washed with deionized water, removed from the bioprocessor and air dried for 20 minutes. A saturated solution of sinapinic acid (0.5 μl) in 50% acetonitrile, 0.5% trifluoroacetic acid was applied to each spot on the chip surfaces. The array surface was allowed to dry for 10 minutes before another application of 0.5 μl of the sinapinic acid solution.

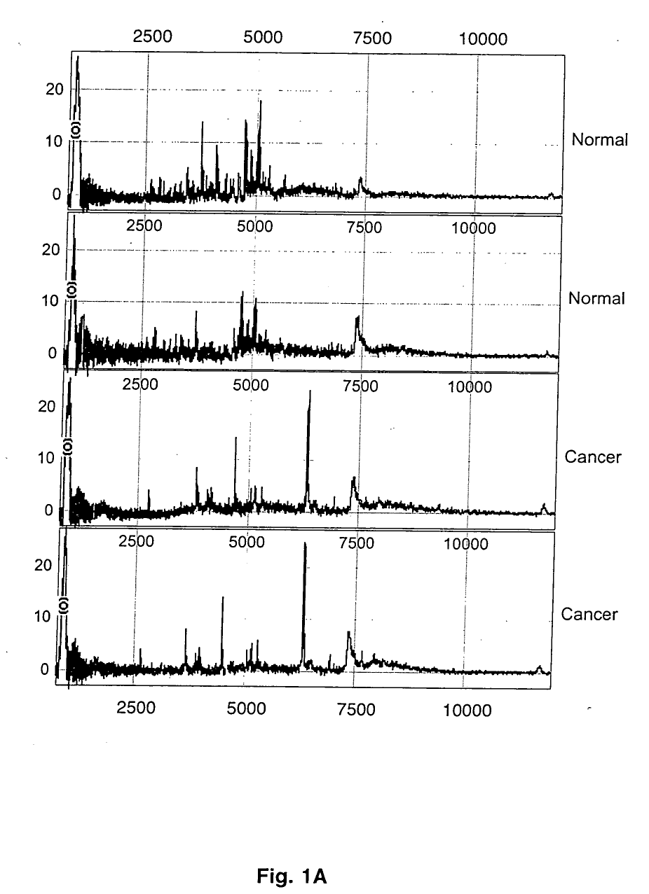

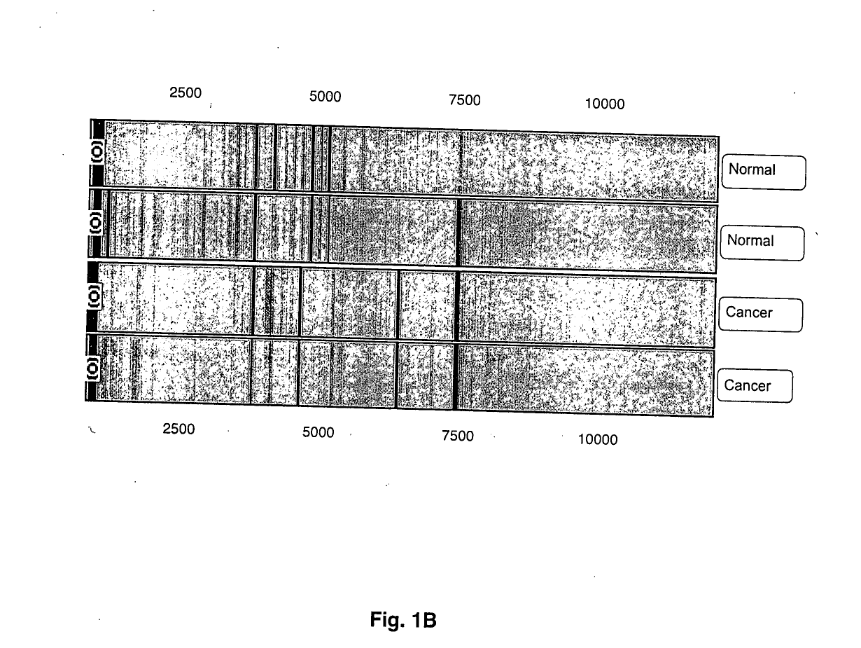

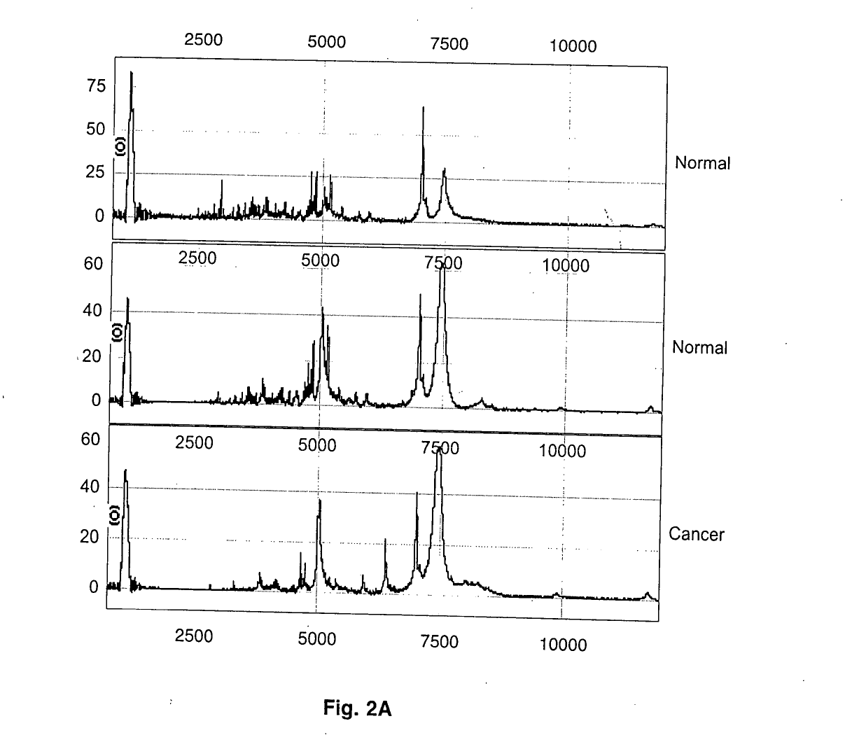

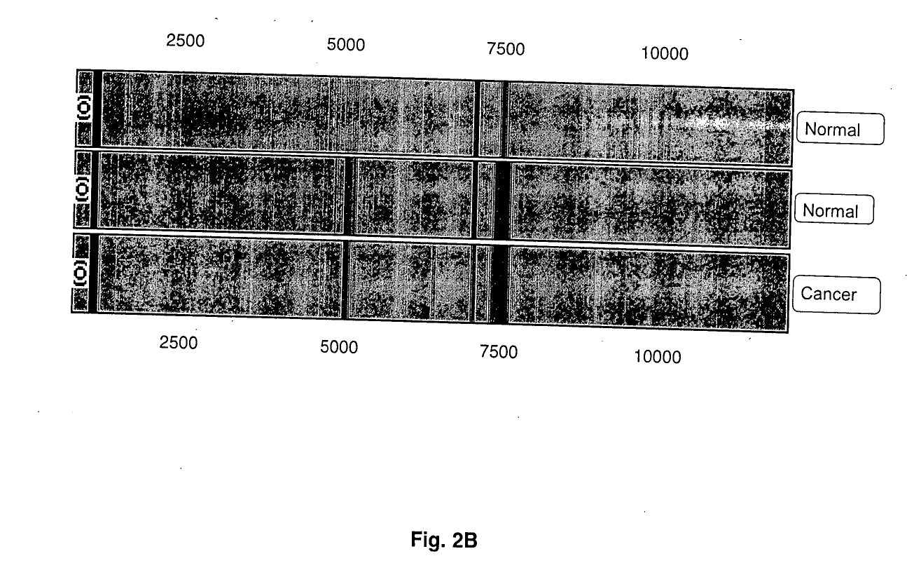

Protein chip arrays were read by a PBS-II C mass analyzer (Ciphergen). The spectral data was acquired using Ciphergen's ProteinChip software version 3.1. The time-of-flight spectra was generated by averaging 156 laser shots in the positive mode with a laser intensity of 180, detector sensitivity of 8 and a focus lag time of 782 ns. The data acquisition parameters were optimized to detect peaks in the range of 1-10 kDa as this range contained the majority of the resolved protein/peptide peaks from preliminary studies. Mass accuracy was calibrated using the All-in-one peptide and All-in-one protein molecular weight standards (Ciphergen). The samples were subjected to surface-enhanced laser desorption/ionization time-of-flight mass spectrometry immediately. The results of the use of surface-enhanced laser desorption/ionization time-of-flight mass spectrometry on tear samples of patients with breast cancer and those without is illustrated in FIGS. 1A-B and 2A-B.

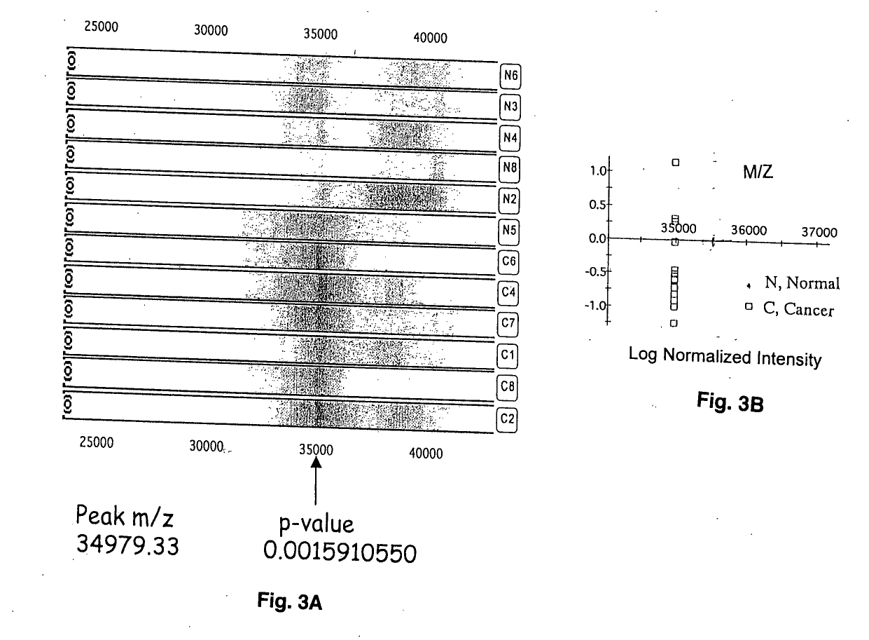

Further studies were conducted with a tear saline wash from 25 patients with benign masses and 25 patients with breast cancer. Tears were taken before and after the lesions were removed. The data acquisition parameters were optimized to detect peaks in the range of 2540 kDa. FIGS. 3A-B demonstrate the identification of a 35 kDa protein found in the tears of almost all breast cancer patients with invasive and non-invasive breast cancer. This protein was also identified in at least one patient with atypia and a strong family history of breast cancer. Three other proteins were found to be present in lower levels in individuals with breast cancer than that present in normal individuals.

### EXAMPLE 3

Statistical Methods

The surface-enhanced laser desorption/ionization time-of-flight mass spectrometry peak intensities obtained by the procedure described above were examined for distributional assumptions and subjected to variance-stabilizing transformations if required. Cases and controls were compared for univariate differences in peak intensities (or transformed values) via a two-sided two-sample t-test. Additionally, this univariate analysis will be performed with multiple-comparison adjustment due to the likelihood of many SELDI peaks. However, the type and stringency of adjustment will be determined in advance by careful consideration of the relative costs of Type I versus Type II error. Univariately significant SELDI peaks will be considered to be candidate biomarkers that have potential to be diagnostic indicators. Multivariate logistic regression will be used to assess groups of SELDI peaks for their collective ability to distinguish cases from controls. To limit overfitting, SELDI peak groups contain no more than five members per multivariate model.

Further, since each peak analyzed has a characteristic variability of unknown magnitude, based on a succinct power analysis it is required to express Case-Control difference as a standardized quantity called Effect Size, in which the difference in group means is expressed as a multiple of the standard deviation (SD) of an individual measurement about its expected value. If the multiple-comparison is via the Bonferroni method, then an adjusted alpha of 0.001 is not unreasonable.

The following references were cited herein:
Ardekani, A. M. et al. (2002) Expert Rev Mol Diagn. 2(4):312-320.
Burtin, T et al. (1998) J Fr Ophtalmol 21(9):637-642.
Dickinson D. N. et al. (2004) J. Microbiol. Methods. 58(1):1-12
Garfinkel, L. et al. (1994) Cancer 74: 222-227.
Luftner, D. et al. (2002) Expert Rev Mol Diagn. 2(1): 23-31.
Miller, T. E. et al. (1992) Cancer Res. 52: 422.
Ries, L. A. G. et al. In SEER Cancer Statistics Review, 1973-1996. National Cancer Institute, Bethesda, Md. 1999. Schulz, B. L. et al. (2002) Biochem J 366(Pt 2): 511-520.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method of identifying proteins useful as a diagnostic indicator of cancer in an individual, comprising:
(a) obtaining a biological sample from said individual;
(b) performing surface-enhanced laser desorption/ionization time-of-flight mass spectrometry on said biological sample;
(c) comparing the protein profile in said sample with a protein profile of control sample, wherein said protein profile of said control sample is prepared using surface-enhanced laser desorption/ionization time-of-flight mass spectrometry; and
selecting proteins in said biological sample having statistically greater peak intensities than the peak intensities of the proteins in said control sample, thereby identifying said proteins as a diagnostic indicator of said cancer.

2. The method of claim 1, wherein said identified proteins have molecular weights of about 1-100 kDa.

3. The method of claim 1, wherein said biological sample and said control sample is tear fluid.

4. The method of claim 1, wherein said control sample is from a healthy individual.

5. The method of claim 1, wherein said proteins are identified in an individual either diagnosed with or suspected of having cancer.

6. The method of claim 5, wherein said cancer is breast cancer.

7. A protein identified by the method of claim 1.

8. The protein of claim 7, wherein said protein has a molecular weight of about 1-100 kDa.

9. The protein of claim 7, wherein said protein is identified in tear fluid.

10. The method of claim 7, wherein said protein is identified in a sample obtained from an individual either diagnosed with or suspected of having cancer.

11. The protein of claim 10, wherein said cancer is breast cancer and wherein said protein has a molecular weight of about 35 kDa.

12. A method of screening an individual for cancer, comprising:
obtaining biological sample from said individual;
determining an amount(s) of protein(s) of claim 7 in said biological sample; and
comparing said amount(s) to the amount(s) of protein(s) in a control sample, wherein increased amount(s) of said protein(s) in said biological sample compared to said control sample indicates that said individual has cancer.

13. The method of claim 12, further comprising:
assessing a response to treatment for cancer in an individual, wherein said assessment comprises:
comparing the amount(s) of protein(s) in the sample of the individual collected post-treatment with the amount(s) of the protein(s) in a control sample or in the sample of the individual collected pre-treatment, wherein the amount(s) of the protein(s) collected post-treatment if same as the amount(s) of the protein(s) in the control sample or decreased compared to the amount(s) of the protein(s) collected pre-treatment indicates that said individual is responsive to said treatment.

15. The method of claim 12, wherein said biological sample and said control sample is tear fluid.

16. The method of claim 12, wherein said control sample is of a healthy individual.

17. The method of claim 12, wherein said individual is suspected of having cancer, at risk of developing cancer or being treated for cancer.

18. The method of claim 17, wherein said cancer is breast cancer.

19. The method of claim 18, wherein a protein of molecular weight of about 35 kDa is present in excess in the tear sample of an individual with said cancer.

20. A method of screening an individual for cancer, comprising:
(a) obtaining a biological sample from said individual;
(b) performing surface-enhanced laser desorption/ionization time-of-flight mass spectrometry on said biological sample;
(c) comparing the protein profile in said sample with a control protein profile typical for said cancer, wherein said protein profile is prepared using surface-enhanced laser desorption/ionization time-of-flight mass spectrometry and wherein a match of the test profile with the control profile indicates that said individual has said cancer.

21. The method of claim 20, wherein said biological sample is a tear sample.

22. The method of claim 20, wherein said control protein profile for said cancer is prepared using a tear sample.

23. The method of claim 20, wherein said tear sample is obtained from an individual diagnosed with said cancer

24. A kit for diagnosis of cancer, wherein the kit comprises proteins identified as diagnostic markers for cancer using the method of claim 1.

25. The kit of claim 24, further comprising antigens and reagents to detect said proteins.
